(19) United States
(12) Patent Application Publication
(10) Pub. No.: US 2026/0167993 A1
(43) Pub. Date: Jun. 18, 2026

(54) METHYLOBACTERIUM EXTORQUENS AND METHOD FOR PRODUCING LACTIC ACID USING SAME

(71) Applicant: CPC CORPORATION, TAIWAN, Kaohsiung City (TW)

(72) Inventors: HSIN-YI LEE, Chiayi City (TW); CHIN-CHUNG CHEN, Chiayi City (TW); ETHAN I LAN, Hsinchu City (TW); YOU-AN CHEN, Zhubei City (TW); BILLY C. HUNG, New Taipei City (TW); YU-CHIEH LI, New Taipei City (TW)

(73) Assignee: CPC CORPORATION, TAIWAN, Kaohsiung City (TW)

(21) Appl. No.: 19/021,238

(22) Filed: Jan. 15, 2025

(30) Foreign Application Priority Data
Dec. 12, 2024 (TW) 113148287

(51) Int. Cl. C12P 7/56; C12N 1/20; C12N 9/04; C12R 1/01

(52) U.S. Cl. CPC C12P 7/56; C12N 1/20; C12N 9/0006; C12Y 101/01027; C12N 2500/32; C12N 2500/74; C12R 2001/01

(57) ABSTRACT

A Methylobacterium extorquens and a method for producing lactic acid using the same is provide. The Methylobacterium extorquens is capable of fermenting methanol to produce lactic acid. The lactic acid production method comprises the following steps: (a) providing Methylobacterium extorquens that can ferment methanol to produce lactic acid; (b) inoculating the Methylobacterium extorquens into a methanol culture medium containing methanol at a concentration of below 2% v/v, the methanol culture medium has a C/N ratio value of 3.5 to 21 and a pH value of pH 4 to pH 9; and (c) allowing the Methylobacterium extorquens to ferment the methanol in the methanol culture medium to produce lactic acid under 20° C. to 30° C. Through the above Methylobacterium extorquens and lactic acid production method, a method for producing lactic acid using non-crop materials is provided.

### CROSS-REFERENCE TO RELATED APPLICATION

This non-provisional application claims priority under 35 U.S.C. § 119 (a) on Patent Application No(s). 113148287 filed in Taiwan, R.O.C. on Dec. 12, 2024, the entire contents of which are hereby incorporated by reference.

### REFERENCE TO A SEQUENCE LIST

Pursuant to 37 CFR § 1.831-835, the instant application contains a computer readable Sequence Listing which has been submitted electronically in XML format and is hereby incorporated by reference in its entirety. Said XML format file, created on Jan. 7, 2025, is named Sequence Listing.xml and is 37.2 kb in size.

### BACKGROUND OF THE INVENTION

### 1. Field of the Invention

The present disclosure relates to a Methylobacterium extorquens and a method for producing lactic acid using the same, and in particular to a Methylobacterium extorquens that can produce lactic acid using methanol as a source of material.

### 2. Description of the Related Art

Lactic acid is an important natural organic acid, which is widely used in various industries. Lactic acid is also currently the main raw material for manufacturing bioplastic polylactic acid. In addition, polylactic acid has the characteristics of high biocompatibility, non-toxicity and non-irritation, allowing it to be used in medical supplies such as surgical sutures. At present, the main lactic acid production method is biological fermentation, which produces lactic acid through microbial fermentation of sugar raw materials such as corn.

### BRIEF SUMMARY OF THE INVENTION

However, most of the sugar raw materials used in the current mainstream biological fermentation methods are food crops. When food crops are appropriated as raw materials for the production of bioplastics such as polylactic acid, the demand for food consumption will be crowded out. Therefore, finding an alternative lactic acid production method that uses other non-food crops as feed sources to produce lactic acid remains a problem to be solved.

An object of the present disclosure is to provide a Methylobacterium extorquens capable of fermenting methanol to produce lactic acid with respect to the above problems. The Methylobacterium extorquens is deposited at Japan National Institute of Technology and Evaluation with a accession number NITE BP-04142.

In order to achieve the above and other objects, a method for producing lactic acid is provided. The method includes the following steps: (a) providing a Methylobacterium extorquens capable of fermenting methanol to produce lactic acid, wherein the Methylobacterium extorquens is deposited at Japan National Institute of Technology and Evaluation with a accession number NITE BP-04142; (b) inoculating the Methylobacterium extorquens in a methanol culture medium containing methanol at a concentration of below 2% v/v, wherein the methanol culture medium has a C/N ratio between 3.5 and 21 and a pH value between pH 4 and pH 9; and (c) allowing the Methylobacterium extorquens to ferment the methanol in the methanol culture medium to produce lactic acid under 20° C. to 30° C.

According to the method as described above, in step (a), the Methylobacterium extorquens is caused to express lactate dehydrogenase gene ldh V39R of Bacillus subtilis in vivo.

According to the method as described above, the concentration of the methanol in the methanol culture medium ranges from 0.5% to 1%.

According to the method as described above, the pH value of the methanol culture medium ranges from pH 7 to pH 8.

According to the method as described above, the C/N ratio of the methanol culture medium ranges from 13 to 15.

According to the method as described above, the methanol culture medium further contains yeast extract and peptone.

By using the Methylobacterium extorquens and the method for producing lactic acid as described above, a lactic acid production method that can produce lactic acid from non-food crop raw materials is provided, thereby solving the problem that the conventional lactic acid production method will crowd out the demand for food consumption.

### BRIEF DESCRIPTION OF THE DRAWINGS

FIG. 1 shows a comparative chart of the effects of knocking out different genes on lactate consumption.

FIG. 2 shows the HPLC spectrum of the fermentation product of Methylobacterium Extorquens according to an embodiment of the present disclosure.

FIG. 3 shows a graph of the lactic acid yield of Methylobacterium Extorquens according to an example of the present disclosure.

FIG. 4 shows the lactic acid yield of Methylobacterium Extorquens under different pH environments according to an embodiment of the present disclosure.

FIG. 5 shows the growth trend of Methylobacterium Extorquens under different pH environments according to an embodiment of the present disclosure.

FIG. 6 shows the long-term lactic acid yield of Methylobacterium Extorquens under a pH 7 environment according to an embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

### Preparation of Methylobacterium Extorquens of the Examples

In the examples, methanol is used as the fermentation raw material for producing lactic acid, and a lactic acid production method that can produce lactic acid from non-food crop raw materials is proposed. Since methanol is a common by-product of the chemical industry chain, coupled with the development of biogas technology in recent years, the current sources of methanol have increased significantly. Therefore, methanol is suitable as a fermentation source to replace food crops. In the examples, Methylobacterium Extorquens among the methanotrophic bacteria is selected as the strain for producing lactic acid. In order to increase the yield of lactic acid produced by Methylobacterium Extorquens and provide a lactic acid production method that can be applied to large-scale production of lactic acid, the Methylobacterium Extorquens BP-04142 of the example further increase its yield of lactic acid by gene knockout method. More specifically, the Methylobacterium Extorquens BP-04142 of the example knocks out the celABC and lldEFG genes that cause lactic acid consumption in the strain (it is currently known that the celABC gene produces cellulose and causes strain aggregation such that the yield of converting methanol into lactic acid is reduced due to the synthesis of cellulose using the carbon flux of the strain. Therefore, knocking out celABC can reduce the lactic acid consumption of the strain), and further increase the strain's lactic acid production by reducing the strain's own lactic acid consumption. In the following series of test results, it is proved that Methylobacterium Extorquens BP-04142 of the example can achieve the expected increase in lactic acid production of the strain. The preparation process of Methylobacterium Extorquens BP-04142 in the example is as what follows.

First, the knockout plasmid required to knock out the specific genes of Methylobacterium Extorquens is constructed. In this example, pCM184 plasmid is used as the skeleton to establish the Cre-loxP recombination system to knock out the target gene. The steps to establish the Cre-loxP recombination system are as what follows. First, the following five gene fragments are amplified by PCR method to construct the first knockout plasmid: the plasmid skeleton (Fragment 1, whose sequence is SEQ ID NO: 1), the homologous sequence at the 5′ end of the gene to be knocked out (Fragment 2) (Fragment 2 of the first knockout plasmid is a fragment for knocking out the celABC gene, and the sequence of Fragment 2 for knocking out the celABC gene is SEQ ID NO: 2), loxP sequence and the upper half of the kanamycin gene (Fragment 3, whose sequence is SEQ ID NO: 4), another loxP sequence and the lower half of the kanamycin gene (Fragment 4, whose sequence is SEQ ID NO: 5) and the homologous sequence at the 3′ end of the gene to be knocked out (Fragment 5) (Fragment 5 of the first knockout plasmid is a fragment for knocking out the celABC gene, and the sequence of Fragment 5 for knocking out the celABC gene is SEQ ID NO: 6). Next, Fragments 2 and 3 are overlapped and joined by PCR, and Fragments 4 and 5 are overlapped and joined by PCR, thereby obtaining two large fragments, i.e. fragment 2/3 and fragment 4/5, both of which are then joined with Fragment 1, and finally the first knockout plasmid for knocking out the celABC gene can be obtained.

At the same time, the following 5 gene fragments are amplified by PCR method to construct the second knockout plasmid: the plasmid skeleton (Fragment 1, whose sequence is SEQ ID NO: 1), the homologous sequence at the 5′ end of the gene to be knocked out (Fragment 2) (Fragment 2 of the second knockout plasmid is a fragment for knocking out the lldEFG gene, and the sequence for knocking out the lldEFG gene is SEQ ID NO: 3), loxP sequence and the upper half of the kanamycin gene (Fragment 3, whose sequence is SEQ ID NO: 4), another loxP sequence and the lower half of the kanamycin gene (Fragment 4, whose sequence is SEQ ID NO: 5) and the homologous sequence at the 3′ end of the gene to be knocked out (Fragment 5) (Fragment 5 of the second knockout plasmid is a fragment for knocking out the lldEFG gene, and the sequence for knocking out the lldEFG gene is SEQ ID NO: 7). Next, Fragments 2 and 3 are overlapped and joined by PCR method, and Fragments 4 and 5 are overlapped and joined by PCR method, thereby obtaining two large fragments, i.e. fragment 2/3 and fragment 4/5, both of which are then joined with Fragment 1, and finally the second knockout plasmid for knocking out the lldEFG gene can be obtained.

The sequences of the aforementioned SEQ ID NO: 1 to SEQ ID NO: 7 are as described in Table 1 below. The primer sequences used to prepare the first knockout plasmid and the second knockout plasmid mentioned above are described in Table 2 below.

Because the Methylobacterium extorquens in this example needs to knock out the celABC gene and the lldEFG gene at the same time, therefore, the following steps for knocking out the celABC gene and lldEFG gene in Methylobacterium extorquens will involve two plasmid transfers, that is, the first knockout plasmid is first transferred into Methylobacterium extorquens to knock out the celABC gene, and then the second knockout plasmid is transferred into Methylobacterium extorquens to knock out the lldEFG gene. The process of knocking out specific genes in Methylobacterium extorquens will be further described below. The process for transferring the first knockout plasmid into Methylobacterium extorquens to knock out the gene is exactly the same as that for the second knockout plasmid.

After the knockout plasmid (the first knockout plasmid and the second knockout plasmid) is constructed, the Methylobacterium extorquens (purchased from German Microbiology and Cell Culture Co., Ltd. (DSMZ Company)) is inoculated into 2 mL of the culture medium C. The culture medium C is prepared by mixing the culture medium A and the culture medium B in a ratio of 1:1. The composition of the culture medium A is as what follows: 5 g/L of peptone, 3 g/L of beef extract, and 1% v/v methanol. The composition of the culture medium B is as what follows: 1 g/L of (NH₄)₂SO₄, 0.448 g/L of MgSO₄·7H₂O, 1.304 g/L of KH₂PO₄, 2.128 g/L of Na₂HPO₄, 0.0032 g/L of CaCl₂), 0.0134 g/L of Na₃C₆H₃O₇·2H₂O, 0.005 g/L of FeSO₄·7H₂O, 0.00034 g/L of ZnSO₄·7H₂O, 0.00018 g/L of MnCl₂·4H₂O, 0.00024 g/L of CuSO₄·5H₂O, 0.00046 g/L of CoCl₂·6H₂O, 0.000028 g/L of H₃BO₃, 0.000484 g/L of Na₂WO₄·2H₂O, 0.000108 g/L of Na₂MoO₄·2H₂O and 1% v/v methanol.

The culture medium C in which the Methylobacterium extorquens is inoculated is placed in a 30° C. incubator and pre-cultured at a rotation speed of 250 rpm for 2 days, and then 0.2 ml of the pre-cultured bacterial liquid is diluted to 1/100 times, added into 20 mL of the culture medium C again and cultured at a rotation speed of 250 rpm at 30° C. for 18 hours. After the culture is completed, the cultured bacterial liquid is centrifuged at 4000 rpm at 4° C. to separate the Methylobacterium extorquens strain from the supernatant. After removing the supernatant, the strain is placed on ice, the cells are washed three times with ice-cold 10% glycerol, and then 1 mL of 10% glycerol is added to re-dissolve the strain.

50 μl of the bacterial liquid are extracted from the aforementioned bacterial liquid re-dissolved with glycerol, and 200 ng of the knockout plasmid are added therein. The bacterial liquid added with the knockout plasmid is placed in a 2 mm electroporation test tube, and the strain is electroporated under the parameters of 2500 V voltage, 200Ω resistance and 25 μF capacitance.

After the electroporation is completed, the electroporation test tube is placed on ice, 200 μL of the culture medium A is added into the electroporation test tube, and then all the bacterial liquid in the electroporation test tube is taken out, added into a test tube containing 800 μL of the culture medium A and cultured at 30° C. and a rotation speed of 250 rpm for 2 hours. After the culture is completed, the bacterial liquid is inoculated into the solid culture medium C (15 g/L agar are added into the liquid culture medium C) containing kanamycin (at a concentration of 50 μg/mL) to screen out the Methylobacterium extorquens strains that have been transferred with the knockout plasmid.

In addition, after the first knockout plasmid is transferred into Methylobacterium extorquens to complete the knockout of the celABC gene, it is necessary to transfer the plasmid pCM157 that can express Cre recombinase into Methylobacterium extorquens, in order to knock out the antibiotic resistance gene introduced by the first knockout plasmid for strain selection in the Methylobacterium extorquens. After the antibiotic resistance gene in the Methylobacterium extorquens has been knocked out, the second knockout plasmid is then transferred into the Methylobacterium extorquens, and the antibiotic resistance gene introduced by the second knockout plasmid in the Methylobacterium extorquens is also knocked out after the second knockout plasmid has been transferred into the Methylobacterium extorquens and the lldEFG gene has been knocked out. After completing the above steps, the Methylobacterium extorquens in which the celABC and lldEFG genes have been knocked out of the example can be obtained.

TABLE 1
Gene fragments used to construct knockout plasmids

| Sequence | |
|---|---|
| No. | Fragment sequence |
| SEQ ID | GGATCCAGCTTATCGATACCGCGGGCCCTACGTACGCGTGT |
| NO: 1 | TAACCGGTGAGCTCACTAGAGGATCCAGCCGACCAGGCTTT |
| | CCACGCCCGCGTGCCGCTCCATGTCGTTCGCGCGGTTCTCG |
| | GAAACGCGCTGCCGCGTTTCGTGATTGTCACGCTCAAGCCC |
| | GTAGTCCCGTTCGAGCGTCGCGCAGAGGTCAGCGAGGGCGC |
| | GGTAGGCCCGATACGGCTCATGGATGGTGTTTCGGGTCGGG |
| | TGAATCTTGTTGATGGCGATATGGATGTGCAGGTTGTCGGT |
| | GTCGTGATGCACGGCACTGACGCGCTGATGCTCGGCGAAGC |
| | CAAGCCCAGCGCAGATGCGGTCCTCAATCGCGCGCAACGTC |
| | TCCGCGTCGGGCTTCTCTCCCGCGCGGAAGCTAACCAGCAG |
| | GTGATAGGTCTTGTCGGCCTCGGAACGGGTGTTGCCGTGCT |
| | GGGTCGCCATCACCTCGGCCATGACAGCGGGCAGGGTGTTT |
| | GCCTCGCAGTTCGTGACGCGCACGTGACCCAGGCGCTCGGT |
| | CTTGCCTTGCTCGTCGGTGATGTACTTCACCAGCTCCGCGAA |
| | GTCGCTCTTCTTGATGGAGCGCATGGGGACGTGCTTGGCAA |
| | TCACGCGCACCCCCCGGCCGTTTTAGCGGCTAAAAAAGTCA |
| | TGGCTCTGCCCTCGGGCGGACCACGCCCATCATGACCTTGC |
| | CAAGCTCGTCCTGCTTCTCTTCGATCTTCGCCAGCAGGGCGA |
| | GGATCGTGGCATCACCGAACCGCGCCGTGCGCGGGTCGTCG |
| | GTGAGCCAGAGTTTCAGCAGGCCGCCCAGGCGGCCCAGGTC |
| | GCCATTGATGCGGGCCAGCTCGCGGACGTGCTCATAGTCCA |
| | CGACGCCCGTGATTTTGTAGCCCTGGCCGACGGCCAGCAGG |
| | TAGGCCGACAGGCTCATGCCGGCCGCCGCCGCCTTTTCCTC |
| | AATCGCTCTTCGTTCGTCTGGAAGGCAGTACACCTTGATAG |
| | GTGGGCTGCCCTTCCTGGTTGGCTTGGTTTCATCAGCCATCC |
| | GCTTGCCCTCATCTGTTACGCCGGCGGTAGCCGGCCAGCCT |
| | CGCAGAGCAGGATTCCCGTTGAGCACCGCCAGGTGCGAATA |
| | AGGGACAGTGAAGAAGGAACACCCGCTCGCGGGTGGGCCT |
| | ACTTCACCTATCCTGCCCGGCTGACGCCGTTGGATACACCA |
| | AGGAAAGTCTACACGAACCCTTTGGCAAAATCCTGTATATC |
| | GTGCGAAAAAGGATGGATATACCGAAAAAATCGCTATAAT |
| | GACCCCGAAGCAGGGTTATGCAGCGGAAAAGCGCTGCTTCC |
| | CTGCTGTTTTGTGGAATATCTACCGACTGGAAACAGGCAAA |
| | TGCAGGAAATTACTGAACTGAGGGGACAGGCGAGAGACGA |
| | TGCCAAAGAGCTACACCGACGAGCTGGCCGAGTGGGTTGA |
| | ATCCCGCGCGGCCAAGAAGCGCCGGCGTGATGAGGCTGCG |
| | GTTGCGTTCCTGGCGGTGAGGGCGGATGTCGAGGCGGCGTT |
| | AGCGTCCGGCTATGCGCTCGTCACCATTTGGGAGCACATGC |
| | GGGAAACGGGGAAGGTCAAGTTCTCCTACGAGACGTTCCGC |
| | TCGCACGCCAGGCGGCACATCAAGGCCAAGCCCGCCGATGT |
| | GCCCGCACCGCAGGCCAAGGCTGCGGAACCCGCGCCGGCA |
| | CCCAAGACGCCGGAGCCACGGCGGCCGAAGCAGGGGGGCA |
| | AGGCTGAAAAGCCGGCCCCCGCTGCGGCCCCGACCGGCTTC |
| | ACCTTCAACCCAACACCGGACAAAAAGGATCCCCAATTCTC |
| | ATGTTTGACAGCTTATCATCGATAAGCTTTAATGCGGTAGTT |
| | TATCACAGTTAAATTGCTAACGCAGTCAGGCACCGTGTATG |
| | AAATCTAACAATGCGCTCATCGTCATCCTCGGCACCGTCAC |
| | CCTGGATGCTGTAGGCATAGGCTTGGTTATGCCGGTACTGC |
| | CGGGCCTCTTGCGGGATATCGTCCATTCCGACAGCATCGCC |
| | AGTCACTATGGCGTGCTGCTAGCGCTATATGCGTTGATGCA |
| | ATTTCTATGCGCACCCGTTCTCGGAGCACTGTCCGACCGCTT |
| | TGGCCGCCGCCCAGTCCTGCTCGCTTCGCTACTTGGAGCCA |
| | CTATCGACTACGCGATCATGGCGACCACACCCGTCCTGTGG |
| | ATCCTCTACGCCGGACGCATCGTGGCCGGCATCACCGGCGC |
| | CACAGGTGCGGTTGCTGGCGCCTATATCGCCGACATCACCG |
| | ATGGGGAAGATCGGGCTCGCCACTTCGGGCTCATGAGCGCT |
| | TGTTTCGGCGTGGGTATGGTGGCAGGCCCCGTGGCCGGGGG |
| | ACTGTTGGGCGCCATCTCCTTGCATGCACCATTCCTTGCGGC |
| | GGCGGTGCTCAACGGCCTCAACCTACTACTGGGCTGCTTCC |
| | TAATGCAGGAGTCGCATAAGGGAGAGCGTCGACCGATGCC |
| | CTTGAGAGCCTTCAACCCAGTCAGCTCCTTCCGGTGGGCGC |
| | GGGGCATGACTATCGTCGCCGCACTTATGACTGTCTTCTTTA |
| | TCATGCAACTCGTAGGACAGGTGCCGGCAGCGCTCTGGGTC |
| | ATTTTCGGCGAGGACCGCTTTCGCTGGAGCGCGACGATGAT |
| | CGGCCTGTCGCTTGCGGTATTCGGAATCTTGCACGCCCTCGC |
| | TCAAGCCTTCGTCACTGGTCCCGCCACCAAACGTTTCGGCG |
| | AGAAGCAGGCCATTATCGCCGGCATGGCGGCCGACGCGCTG |
| | GGCTACGTCTTGCTGGCGTTCGCGACGCGAGGCTGGATGGC |
| | CTTCCCCATTATGATTCTTCTCGCTTCCGGCGGCATCGGGAT |
| | GCCCGCGTTGCAGGCCATGCTGTCCAGGCAGGTAGATGACG |
| | ACCATCAGGGACAGCTTCAAGGATCGCTCGCGGCTCTTACC |
| | AGCCTAACTTCGATCACTGGACCGCTGATCGTCACGGCGAT |
| | TTATGCCGCCTCGGCGAGCACATGGAACGGGTTGGCATGGA |
| | TTGTAGGCGCCGCCCTATACCTTGTCTGCCTCCCCGCGTTGC |
| | GTCGCGGTGCATGGAGCCGGGCCACCTCGACCTGAATGGAA |
| | GCCGGCGGCACCTCGCTAACGGATTCACCACTCCAAGAATT |
| | GGAGCCAATCAATTCTTGCGGAGAACTGTGAATGCGCAAAC |
| | CAACCCTTGGCAGAACATATCCATCGCGTCCGCCATCTCCA |
| | GCAGCCGCACGCGGCGCATCTCGGGCAGCGTTGGGTCCTGG |
| | CCACGGGTGCGCATGATCGTGCTCCTGTCGTTGAGGACCCG |
| | GCTAGGCTGGCGGGGTTGCCTTACTGGTTAGCAGAATGAAT |
| | CACCGATACGCGAGCGAACGTGAAGCGACTGCTGCTGCAA |
| | AACGTCTGCGACCTGAGCAACAACATGAATGGTCTTCGGTT |
| | TCCGTGTTTCGTAAAGTCTGGAAACGCGGAAGTCAGCGCTC |
| | TTCCGCTTCCTCGCTCACTGACTCGCTGCGCTCGGTCGTTCG |
| | GCTGCGGCGAGCGGTATCAGCTCACTCAAAGGCGGTAATAC |
| | GGTTATCCACAGAATCAGGGGATAACGCAGGAAAGAACAT |
| | GTGAGCAAAAGGCCAGCAAAAGGCCAGGAACCGTAAAAAG |
| | GCCGCGTTGCTGGCGTTTTTCCATAGGCTCCGCCCCCCTGAC |
| | GAGCATCACAAAAATCGACGCTCAAGTCAGAGGTGGCGAA |
| | ACCCGACAGGACTATAAAGATACCAGGCGTTTCCCCCTGGA |
| | AGCTCCCTCGTGCGCTCTCCTGTTCCGACCCTGCCGCTTACC |
| | GGATACCTGTCCGCCTTTCTCCCTTCGGGAAGCGTGGCGCTT |
| | TCTCATAGCTCACGCTGTAGGTATCTCAGTTCGGTGTAGGTC |
| | GTTCGCTCCAAGCTGGGCTGTGTGCACGAACCCCCCGTTCA |
| | GCCCGACCGCTGCGCCTTATCCGGTAACTATCGTCTTGAGTC |
| | CAACCCGGTAAGACACGACTTATCGCCACTGGCAGCAGCCA |
| | CTGGTAACAGGATTAGCAGAGCGAGGTATGTAGGCGGTGCT |
| | ACAGAGTTCTTGAAGTGGTGGCCTAACTACGGCTACACTAG |
| | AAGGACAGTATTTGGTATCTGCGCTCTGCTGAAGCCAGTTA |
| | CCTTCGGAAAAAGAGTTGGTAGCTCTTGATCCGGCAAACAA |
| | ACCACCGCTGGTAGCGGTGGTTTTTTTGTTTGCAAGCAGCA |
| | GATTACGCGCAGAAAAAAAGGATCTCAAGAAGATCCTTTGA |
| | TCTTTTCTACGGGGTCTGACGCTCAGTGGAACGAAAACTCA |
| | CGTTAAGGGATTTTGGTCATGAGATTATCAAAAAGGATCTT |
| | CACCTAGATCCTTTTAAATTAAAAATGAAGTTTTAAATCAA |
| | TCTAAAGTATATATGAGTAAACTTGGTCTGACAGTTACCAA |
| | TGCTTAATCAGTGAGGCACCTATCTCAGCGATCTGTCTATTT |
| | CGTTCATCCATAGTTGCCTGACTCCCCGTCGTGTAGATAACT |
| | ACGATACGGGAGGGCTTACCATCTGGCCCCAGTGCTGCAAT |
| | GATACCGCGAGACCCACGCTCACCGGCTCCAGATTTATCAG |
| | CAATAAACCAGCCAGCCGGAAGGGCCGAGCGCAGAAGTGG |
| | TCCTGCAACTTTATCCGCCTCCATCCAGTCTATTAATTGTTG |
| | CCGGGAAGCTAGAGTAAGTAGTTCGCCAGTTAATAGTTTGC |
| | GCAACGTTGTTGCCATTGCTACAGGCATCGTGGTGTCACGC |
| | TCGTCGTTTGGTATGGCTTCATTCAGCTCCGGTTCCCAACGA |
| | TCAAGGCGAGTTACATGATCCCCCATGTTGTGCAAAAAAGC |
| | GGTTAGCTCCTTCGGTCCTCCGATCGTTGTCAGAAGTAAGTT |
| | GGCCGCAGTGTTATCACTCATGGTTATGGCAGCACTGCATA |
| | ATTCTCTTACTGTCATGCCATCCGTAAGATGCTTTTCTGTGA |
| | CTGGTGAGTACTCAACCAAGTCATTCTGAGAATAGTGTATG |
| | CGGCGACCGAGTTGCTCTTGCCCGGCGTCAATACGGGATAA |
| | TACCGCGCCACATAGCAGAACTTTAAAAGTGCTCATCATTG |
| | GAAAACGTTCTTCGGGGCGAAAACTCTCAAGGATCTTACCG |
| | CTGTTGAGATCCAGTTCGATGTAACCCACTCGTGCACCCAA |
| | CTGATCTTCAGCATCTTTTACTTTCACCAGCGTTTCTGGGTG |
| | AGCAAAAACAGGAAGGCAAAATGCCGCAAAAAAGGGAATA |
| | AGGGCGACACGGAAATGTTGAATACTCATACTCTTCCTTTTT |
| | CAATATTATTGAAGCATTTATCAGGGTTATTGTCTCATGAGC |
| | GGATACATATTTGAATGTATTTAGAAAAATAAACAAATAGG |
| | GGTTCCGCGCACATTTCCCCGAAAAGTGCCACCTGACGTCT |
| | AGATCTGAATTCAGCTGTACAATTGGTACCATGGATGCATA |
| | TGGCGGCCGC |
| SEQ ID | TACACCGGCGCGCAGGCGCAGGCGGTGGACGACAGCTGGG |
| NO: 2 | CGGTGGCCGTCGATGGCTCGACCAGCATCACCTCCAATTCC |
| | TCGGCCTTCGGCTCCGTCACCGTGACGACGGCACTCGGCAG |
| | CCCGCCGACGGTGAGCGGCGCGCGCGTGCGCGTCGAGGCC |
| | GTGGCCGGCACCTACTCCTATCCCGGACAAGGCGTCGGCGC |
| | CCGCGTCACCGGCTACCAGCAGGAGGGCTCGCTGCTCACCG |
| | GCTACGAGTGGATCTGGCGGGAGGCGGCACTCGCCGGCTAT |
| | GTCGGCTTCAACGTCCGCAGCAACCAGCTCTCGATCCCCGA |
| | CCCCGGCAACCCGGTCGTCGGAACGGGAGTGGGCCTGAAG |
| | GTCGCCGGCAACTTCTACGCAACGCCGACCGACCGGACCAT |
| | GGTCTCGGCCTACGGCTCCTACTCGACCAAGTTCAACGCCT |
| | ACTATTCCCGTTTCCGCGTCGGCTACATGGTCGCCGACGGC |
| | GTCTATATCGGCCCGGAGGCGCTGTTCCTCGGCGACGACTT |
| | CTTCCGCCAGTACCGCGTCGGCGCCCATCTCTCGGGTTTGA |
| | GCTTCGGCCCCGTCCAGATGTCGCTCGCGGCGGGCTACGTG |
| | CGGGACCGCGTCCAGGGCACGGGCTACTATTCCAGCATCGA |
| | GGCGCGCGCGAACTTTTAGAGCGCATTCCGACGGCGAGCAG |
| | GCCGCCGCTCGCGGCGGCGGCCGAAGTCAAGCGCTTGGATC |
| | AATGCGGACAATCCGCCGCCTTGTCTCCGCGTCTCACGGCA |
| | AAGTACTGCTGTTCCAAGCGAAGAATGGCCCTCATATCAGG |
| | AAGACATCCCTCGCCATCGCATGTCATTTCCGCGAAATTCCT |
| | GCCGAATCTTCGGACATTTTCTACGGATGACGCTACGCTCT |
| | GCATCGCAAAATCAACCTTGTTCGCAGAGCGGGACGGCGGC |
| | GTCGTGAACGCATCCGGAAATCGTCGTATTTTAATGATGGA |
| | TCCGTCCACGAGGCAGGG |
| SEQ ID | CGGCGGAAGAAAGCGCGTTGACACTAAGACCTAGAGACGC |
| NO: 3 | CGGCCTGATGCAATCAGGTCGGATACGGCTCTAGGACGATC |
| | GATGGGACGAGCACTCTGAGACCCGCCATGGAATTCGGACC |
| | TTACGCCCCCTTCATCGTCGGTTCCTACGGCTTCGCGGCCCT |
| | GGTGCTCGGCGCCCTGGCCCTCAACGGCTGGTGGGATGGGC |
| | GGACGCAGCGCCGCGCGCTCGCCGAGCTGCAGGACGAGCG |
| | GGGAGGTCGCTCGTGAGCGCGCAGATCCCGCACGCGACCCC |
| | GCCCGGTACCGGGGAGCGCCCCCGCCGCTCGCTCCTCGCGC |
| | TGCTACCGGTGCTCGTCTTCGCCATCTTGGCCGTCGCCTTCC |
| | TGGTCCGGCTGCGCTCGGGCGCCGATCCTGCGGCTTTGCCC |
| | TCGGCGTTGATCGGCAAACCGGTCCCCGCTTTCGCGCTGGA |
| | GCCGGTGCCGGGCCTCCAGGCCGCCGGCCGGCCGGTGCCCG |
| | GTTTGTCGAGTGCCGACCTCAAGGGGCAGGTGACCGTCCTC |
| | AACATCTTCGCCTCGTGGTGCGCCCCCTGCCAGATCGAGCA |
| | TCCGATGCTGATGCGGCTCGCGCAGGAGCCCGGCATCCGCC |
| | TCGTCGGCATCGACTACAAGGATCCCGGCGACGCCGGACGG |
| | CGCTGGCTGGAGCGCAACGGCCTGCCGTTCGCGGCGGTCGG |
| | CGCCGACACGAGCGGCCGGGCCGGAATCGACCTCGGCGTCT |
| | ACGGCGTGCCGGAGACCTTCATCATCGGTCCCGAAGGCCGC |
| | ATCCGCGACAAGCTCGTCGGAATCCTGACGCCGGAAAACTA |
| | CGCGAGCGTGCTGGAGCGTATTCGCGCCGCCGGACGGACCG |
| | CCCCCGTGACCAACTGACCGGGATCAGGCGGCACGCTTGGA |
| | AACCGGGGATCGGAGGCGTATGCAGCCCCTGTCCGCTTGAA |
| | ACCGTTCCAAGGTGGAGAAGCGGGCCAACCGTCTCAAATCA |
| | CGATCAGAAGAGCGCC |
| SEQ ID | ATAACTTCGTATAGCATACATTATACGAAGTTATCTAGACC |
| NO: 4 | TGCAGGGGGGGGGGGGAAAGCCACGTTGTGTCTCAAAATCT |
| | CTGATGTTACATTGCACAAGATAAAAATATATCATCATGAA |
| | CAATAAAACTGTCTGCTTACATAAACAGTAATACAAGGGGT |
| | GTTATGAGCCATATTCAACGGGAAACGTCTTGCTCGAGGCC |
| | GCGATTAAATTCCAACATGGATGCTGATTTATATGGGTATA |
| | AATGGGCTCGCGATAATGTCGGGCAATCAGGTGCGACAATC |
| | TATCGATTGTATGGGAAGCCCGATGCGCCAGAGTTGTTTCT |
| | GAAACATGGCAAAGGTAGCGTTGCCAATGATGTTACAGATG |
| | AGATGGTCAGACTAAACTGGCTGACGGAATTTATGCCTCTT |
| | CCGACCATCAAGCATTTTATCCGTACTCCTGATGATGCATG |
| | GTTACTCACCACTGCGATCCCCGGGAAAACAGCATTCCAGG |
| | TATTAGAAGAATATCCTGATTCAGGTGAAAATATTGTTGAT |
| | GCGCTGGCAGTGTTCCTGCGCCGGTTGCATTCGATTCCTGTT |
| | TGTAATTGT |
| SEQ ID | GTTGCATTCGATTCCTGTTTGTAATTGTCCTTTTAACAGCGA |
| NO: 5 | TCGCGTATTTCGTCTCGCTCAGGCGCAATCACGAATGAATA |
| | ACGGTTTGGTTGATGCGAGTGATTTTGATGACGAGCGTAAT |
| | GGCTGGCCTGTTGAACAAGTCTGGAAAGAAATGCATAAGCT |
| | TTTGCCATTCTCACCGGATTCAGTCGTCACTCATGGTGATTT |
| | CTCACTTGATAACCTTATTTTTGACGAGGGGAAATTAATAG |
| | GTTGTATTGATGTTGGACGAGTCGGAATCGCAGACCGATAC |
| | CAGGATCTTGCCATCCTATGGAACTGCCTCGGTGAGTTTTCT |
| | CCTTCATTACAGAAACGGCTTTTTCAAAAATATGGTATTGAT |
| | AATCCTGATATGAATAAATTGCAGTTTCATTTGATGCTCGAT |
| | GAGTTTTTCTAATCAGAATTGGTTAATTGGTTGTAACACTGG |
| | CAGAGCATTACGCTGACTTGACGGGACGGCGGCTTTGTTGA |
| | ATAAATCGAACTTTTGCTGAGTTGAAGGATCAGATCACGCA |
| | TCTTCCCGACAACGCAGACCGTTCCGTGGCAAAGCAAAAGT |
| | TCAAAATCACCAACTGGTCCACCTACAACAAAGCTCTCATC |
| | AACCGTGGCTCCCTCACTTTCTGGCTGGATGATGGGGCGAT |
| | TCAGGCCTGGTATGAGTCAGCAACACCTTCTTCACGAGGCA |
| | GACCTCAGCGCCCCCCCCCCCCTGCAGGTCCTAGATAACTT |
| | CGTATAGCATACATTATACGAAGTTAT |
| SEQ ID | GATCGCGTCTTCTCGGCCTCCTCGCGGTACTCCCGGCCCTGC |
| NO: 6 | TCGTCGTCGGCCCGGCCCTGGCGCAGACCGCGCCGGTCCTG |
| | CGCCTGCCCGGCGCCGGTCCGGTCGCGGGCGTCGTCGAGAC |
| | GCGGGTGCGCGATGGGTTCGACCAGCGCATCCGCTTCGAGG |
| | GCGGCGACGGCCGCAACCATGCCGAGGTAGCCCTGCGCAA |
| | CGAGACCGGCGATCTCCTGCTCGCGTTCCCGCCGCGGCTGG |
| | CCAAGCCGAGCCAGTTCGGCATCGACGGCGAGATCGCGGTC |
| | CGCTTTCCGGGGCAGGTCCTGCGCGTGGTCGCGCCGCGGCG |
| | CAACGGCTACGGGCCGATCGGCCTCGCGCTCGGGACCGACT |
| | GCCTCTACGCGTGGCAATGGTTCGAGCGGGCGAGCGCGGTC |
| | CAGCGCGCCGCGAGCCGCGGCGGCCTGTTCGACACGTCGGT |
| | CTCGCCAACCGCGGGCCGGCGCGCCCTCTCCCTCCGGATTC |
| | GGCTGTGCCGCACGGCGCAGGCGAGTCTCGACGATCTCGTC |
| | GCCGCCATCGAGCGGATGACCATCTCTCTTCCCGGCGAGCC |
| | CGGAGCACGGTCCGCTCCCGCGCGACCACGTCCCTCCGCCG |
| | TCCGGCGCGAAAGACCCGCGCCCGTGAAGCGGCAGGCCGC |
| | GAGGCCCACCCCCAGCGAGCCGCCCGCCGAGGCTGCACCG |
| | AAGCCAGCACCGGCCGCCCCGCCGCAGGCCGCGCCGCCGCT |
| | CCGTCCGCCGACGCCAACGGCGCCGGACAGCGGCGGCCGC |
| | CGCTACCTCGCCCCAACGACGCCGCAGGGCGCCGCCCCGGC |
| | CTCCGAACCGCCGGTCGCGAGTCCGGCGCCGGGAGGGACG |
| | CAGCGCTACATCACCGATGTGCCGAAACCGGCAGAGGGCG |
| | CGGGCGGCGGCCTGATGCCGAGCCCGGCTCCGGGCCGCTCG |
| | ACCGGCGAGAGCCTGTCGCGCGATCTCCCCGCCGAGGCGTA |
| | CAAGCCCGCCCCAAGCCGCGA |
| SEQ ID | AAGCGGAGCCGAACGGATCTCTGCGCGTTTGCGGCAAAACG |
| NO: 7 | CCTCGCTCGAACGGGGCGTCTTCCCGCAAAATTCGGAGCCG |
| | TCCCCGTGCGATTCAAGCTCGGTTGCAGTCTTGCCTATGAG |
| | GTCAAGGCGCCCACCACCTTCATCTTCAATGTCGAGGTCGC |
| | GAAGCTGCAAAGCCTCGACATCGCCCGCGAAAGCCTGACCC |
| | TGGCGCCGGACCTGGCGCGGCGCGTCTACGTCACGCCGGAC |
| | CTCAAGAACCGCTATCTCGGCGTGAACGTGCAGCCGGGCCC |
| | GTTCTCGCTCGAATACAATGCCGAGGTGGATCTGGCCGTCG |
| | TGCGGGCCGATCCTGCGACGATCTCCGAGACGCCGGTCTCC |
| | GAACTCCCCCTCGACATCCTCCCCTTCCTGCTGCCGAGCCGC |
| | TTCGTCTCGTCGGACCGGCTCGCCGCCTTCGCCCAAGCCGA |
| | GTTCGGCGACCTTCCGAAGGGCCACCAGCGCGTCAACGCGA |
| | TCTGCAACTGGATCCATGACAACATCGCCTATCGGCGTGGT |
| | TCGAGCGACGGCGAGACGACGGCCGACGAGAGCCTGCTGA |
| | TGCGTGCCGGCGTGTGCCGCGACTTCGCCCATCTCGGCGCC |
| | GCCTTCTGCCGCGGGCTCGGCATCCCGGCCCGGTTCGTGAG |
| | CTGCTACGCCTACGGCCTCGTGCCCAGCGACTTCCACGCCG |
| | TGTTCGAGGCCTATCTCGATGGGCGCTGGTGGCTGTTCGAC |
| | GCGACGCGCCAAGCCCATCTCGACGGTCTCGTGCGCATCGG |
| | CATCGGGCGGGATGCCGCCGAAATCGCCTTCTCGACGCCCT |
| | ACGGCGAGATGCAGCCGACCGGTATGGAGATCCGCATCGAT |
| | CGCGCCGACGGAGGCCCGGAGCCGATGGAGCGCACGGTCG |
| | ATGCGATCTCGACGGAGGAGCCGGCTCCGCATGCCGGCGCG |
| | GCCTGACCGAGGGGCCTCACGATCGACGCCCTACCCGGCGA |
| | ATCCGGTGGCCCGAGCG |

TABLE 2
Primer sequences of knockout plasmids

| | | | Use of |
|---|---|---|---|
| Sequence No. | Primer name | Primer sequence | primer |
| SEQ ID NO: 8 | TT35-pCM184- | GGATCCAGCTTATCGATACC | For |
| | vect-F | GCGGG | knocking |
| SEQ ID NO: 9 | TT36-pCM184- | GCGGCCGCCATATGCATCCA | out |
| | vect-R | TGGTA | plasmid |
| | | | skeleton |
| SEQ ID NO: 10 | TT39-loxp-F | ATAACTTCGTATAGCATACA | For loxP- |
| | | TTATACGAAGTTATCTAGAC | kan^{R} |
| | | C | upstream |
| SEQ ID NO: 11 | TT40-kan-R | ACAATTACAAACAGGAATCG | |
| | | AATGCAAC | |
| SEQ ID NO: 12 | TT41-kan-F | GTTGCATTCGATTCCTGTTTG | For loxP- |
| | | TAATTGT | kan^{R} |
| SEQ ID NO: 13 | TT42-loxp-R | ATAACTTCGTATAATGTATG | downstream |
| | | CTATACGAAGTTATCTAGG | |
| SEQ ID NO: 14 | TT43-celAup-F | TGGATGCATATGGCGGCCGC | For |
| | | TACACCGGCGCGCAGGCGCA | celABC |
| | | GGCGG | upstream |
| SEQ ID NO: 15 | TT44-celAup-R | TGTATGCTATACGAAGTTAT | |
| | | CCCTGCCTCGTGGACGGATC | |
| | | CATCA | |
| SEQ ID NO: 16 | TT45-celCdown- | CATACATTATACGAAGTTAT | For |
| | F | GATCGCGTCTTCTCGGCCTC | celABC |
| | | CTCGC | downstream |
| SEQ ID NO: 17 | TT46-celCdown- | GGTATCGATAAGCTGGATCC | |
| | R | TCGCGGCTTGGGGCGGGCTT | |
| | | GTACG | |
| SEQ ID NO: 18 | TT83-LldEFG | TGGATGCATATGGCGGCCGC | For |
| | up-F | CGGCGGAAGAAAGCGCGTT | lldEFG |
| SEQ ID NO: 19 | TT84-LIdEFG | TGTATGCTATACGAAGTTAT | upstream |
| | up-R | GGCGCTCTTCTGATCGTGAT | |
| | | TTG | |
| SEQ ID NO: 20 | TT85-LIdEFG | CATACATTATACGAAGTTAT | For |
| | down-F | AAGCGGAGCCGAACGGATC | lldEFG |
| | | T | downstream |
| SEQ ID NO: 21 | TT86-LIdEFG | GGTATCGATAAGCTGGATCC | |
| | down-R | CGCTCGGGCCACCGG | |

Deposition of Methylobacterium extorquens

The Methylobacterium extorquens in which the celABC and lldEFG genes have been knocked out in the example is deposited at Japan National Institute of Technology and Evaluation with the accession number NITE BP-04142. In the following, the NITE BP-04142 strain will be used to represent the Methylobacterium extorquens of the example.

Test of the Impact of Different Genes on the Lactic Acid Consumption of the Methylobacterium extorquens

The NITE BP-04142 strain in this example further improves the strain's lactic acid production through reduction of the strain's own lactic acid consumption by knocking out the celABC and lldEFG genes that cause the strain to consume lactic acid in the strain. This test will test the effects of different genes on the lactic acid consumption of the Methylobacterium extorquens. The test process is as what follows.

First, according to the aforementioned process of knocking out specific genes in the Methylobacterium extorquens by knocking out plasmids, the strains, in which specific genes were knocked out, of the following Experimental groups 1-4 and Control group were prepared: the strain of Control group was a wild-type Methylobacterium extorquens; the strain of Experimental group 1 was knocked out celABC only; the strain of Experimental group 2 was knocked out celABC and ldhA; the strain of Experimental group 3 was knocked out celABC and lldEFG (that is, the strain of this example); the strain of Experimental group 4 was knocked out celABC and Mex_1p4596; and the strain of Experimental group 5 was knocked out of celABC and Mex_1p3705.

Next, the strains of Experimental groups 1-5 and Control group were respectively cultured in the solid culture medium C (adding 15 g/L agar to the liquid culture medium C), and a colony of each of Experimental groups 1-5 and Control group was picked from the aforementioned solid culture medium C, inoculated into 2 mL of the culture medium C, and cultured in the shake flask with a rotation speed of 250 rpm at 30° C. for 2 days. After 2 days of culture in the shake flask, the cultured bacterial liquid of Experimental group 1-5 and Control group was diluted to 1/100 times, and then the diluted bacterial liquid of Experimental group 1-5 and Control group was inoculated in the culture medium B containing 10 mM lactic acid, and cultured in the shake flask with a rotation speed of 150 rpm. When each group of strains was just inoculated (at the beginning) and after 24 hours of culture, bacterial fluid samples from Experimental groups 1-5 and Control group were taken and then centrifuged to separate the cell precipitates from the supernatants respectively. Finally, the supernatants of the samples from Experimental groups 1-5 and Control group were extracted and analyzed by using the liquid chromatography Shimadzu Prominence-I (LC2030C 3D) to obtain the remaining amounts of lactic acid in the supernatants at the beginning and the 24th hour respectively. The difference between the remaining lactic acid amounts of Experimental groups 1-5 and Control group at the beginning and the 24th hour was the lactic acid consumption of each group. The aforementioned liquid chromatography Shimadzu Prominence-I (LC2030C 3D) was used with an Agilent HiPlex-H (700×7.7 mm) organic acid analysis column to analyze the lactic acid content of each group.

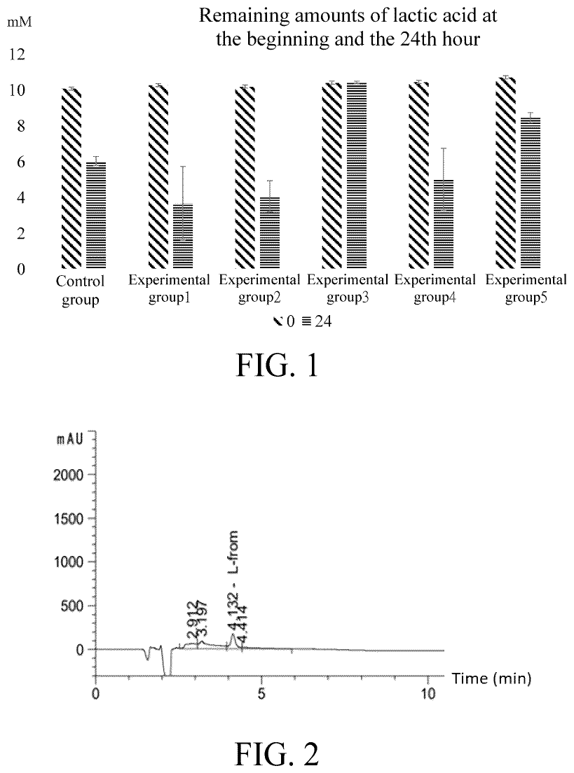

The experimental results are as shown in FIG. 1. Compared with Control group, the lactic acid consumption of Experimental groups 1, 2, and 4 were higher than that of Control group, and Experimental group 3, i.e, the Methylobacterium extorquens, whose celABC and lldEFG genes have been knocked out, had the lowest consumption. It can be seen from the experimental results that the NITE BP-04142 strain of the example, whose celABC and lldEFG genes have been knocked out, can avoid the consumption of lactic acid and prevent the lactic acid produced from being consumed by itself.

In addition, the HPLC spectrum obtained by analyzing the sample of Experimental group 3 with the aforementioned liquid chromatography Shimadzu Prominence-I (LC2030C 3D) is as shown in FIG. 2. The position pointed by the arrow in FIG. 2 represents lactic acid.

Test of Lactic Acid Yield of the Methylobacterium extorquens with Addition of Mutant Lactate Dehydrogenase Gene ldh V39R

It is currently known that adding the mutant lactate dehydrogenase gene ldh V39R to the lactic acid-producing strain can help improve the lactic acid yield of the strain. This test will compare the lactate yield of the NITE BP-04142 strain of this example with the wild-type Methylobacterium extorquens without knocking out the celABC and lldEFG genes with expression of the mutant lactate dehydrogenase gene ldh V39R from Bacillus subtilis. The testing process proceeds as what follows.

First, the wild-type Methylobacterium extorquens was prepared as a control group (into which the anti-kanamycin gene was transferred), and the mutant lactate dehydrogenase gene ldh V39R was transformed into the strain inside the wild-type Methylobacterium extorquens through the plasmid pYS22 (pCM66T plasmid was purchased from addgene company to serve as the cytoskeleton for self-construction, the sequence of the plasmid pYS22 was shown in Table 3 below, and the plasmid pYS22 carried the anti-kanamycin gene) to serve as Experimental group 1. The NITE BP-04142 strain, into which the mutant lactate dehydrogenase gene ldh V39R was transferred through the plasmid pYS22, was used as Experimental group 2. The above-mentioned method of transferring the mutant lactate dehydrogenase gene ldh V39R into the strain referred to the gene transfer method in the process of knocking out specific genes in Methylobacterium extorquens by knocking out plasmids as described above.

Next, the strains of Control group, Experimental group 1 and Experimental group 2 were inoculated into 2 mL of the culture medium C, and pre-cultured at a rotation speed of 250 rpm at 30° C. for 2 to 3 days. After the pre-culture was completed, the bacterial liquid of Control group, Experimental group 1 and Experimental group 2 was inoculated into 20 mL the culture medium B containing 50 μg/mL kanamycin, and the initial OD₆₀₀ value of the fermentation liquid of Control group, Experimental group 1 and Experimental group 2 was adjusted to 0.1. Control group, Experimental group 1 and Experimental group 2 were supplemented with 1% v/v methanol after 12 hours of culture, and samples were taken after 24 hours of culture. The samples of Control group, Experimental group 1 and Experimental group 2 were analyzed for lactic acid content in each group by using the aforementioned liquid chromatography Shimadzu Prominence-I (LC2030C 3D) in conjunction with the Agilent HiPlex-H (700×7.7 mm) organic acid analysis column.

TABLE 3
Sequence of the plasmid pYS22

gaccctttccgacgctcaccgggctggttgccctcgccgctgggctggcggccgtctatggccctgcaaacgc
gccagaaacgccgtcgaagccgtgtgcgagacaccgcggccgccggcgttgtggatacctcgcggaaaact
tggccctcactgacagatgaggggcggacgttgacacttgaggggccgactcacccggcgcggcgttgaca
gatgaggggcaggctcgatttcggccggcgacgtggagctggccagcctcgcaaatcggcgaaaacgcctg
attttacgcgagtttcccacagatgatgtggacaagcctggggataagtgccctgcggtattgacacttgaggg
gcgcgactactgacagatgaggggcgcgatccttgacacttgaggggcagagtgctgacagatgaggggcg
cacctattgacatttgaggggctgtccacaggcagaaaatccagcatttgcaagggtttccgcccgtttttcggc
caccgctaacctgtcttttaacctgcttttaaaccaatatttataaaccttgtttttaaccagggctgcgccctgtgcg
cgtgaccgcgcacgccgaaggggggtgcccccccttctcgaaccctcccggcccgctaacgcgggcctccc
atccccccaggggctgcgcccctcggccgcgaacggcctcaccccaaaaatggcagccaagctgaccacttc
tgcgctcggcccttccggctggctggtttattgctgataaatctggagccggtgagcgtgggtctcgcggtatca
ttgcagcactggggccagatggtaagccctcccgtatcgtagttatctacacgacggggagtcaggcaactatg
gatgaacgaaatagacagatcgctgagataggtgcctcactgattaagcattggtaactgtcagaccaagtttac
tcatatatactttagattgatttaaaacttcatttttaatttaaaaggatctaggtgaagatcctttttgataatctcatg
accaaaatcccttaacgtgagttttcgttccactgagcgtcagaccccgtagaaaagatcaaaggatcttcttga
gatcctttttttctgcgcgtaatctgctgcttgcaaacaaaaaaaccaccgctaccagcggtggtttgtttgccgga
tcaagagctaccaactctttttccgaaggtaactggcttcagcagagcgcagataccaaatactgttcttctagtg
tagccgtagttaggccaccacttcaagaactctgtagcaccgcctacatacctcgctctgctaatcctgttaccag
tggctgctgccagtggcgataagtcgtgtcttaccgggttggactcaagacgatagttaccggataaggcgcag
cggtcgggctgaacggggggttcgtgcacacagcccagcttggagcgaacgacctacaccgaactgagata
cctacagcgtgagctatgagaaagcgccacgcttcccgaagggagaaaggcggacaggtatccggtaagcg
gcagggtcggaacaggagagcgcacgagggagcttccagggggaaacgcctggtatctttatagtcctgtcg
ggtttcgccacctctgacttgagcgtcgatttttgtgatgctcgtcaggggggcggagcctatggaaaaacgcc
agcaacgcggcctttttacggttcctggccttttgctggccttttgctcacatgttctttcctgcgttatcccctgatt
ctgtggataaccgtattaccgcctttgagtgagctgataccgctcgccgcagccgaacgaccgagcgcagcga
gtcagtgagcgaggaagcggaagagcgcccaatacgcaaaccgcctctccccgcgcgttggccgattcatta
atgcagctggcacgacaggtttcccgactggaaagcgggcagtgagcgcaacgcaattaatgtgagttagctc
actcattaggcaccccaggctcccgcttggtcgggccgcttcgcgagggcccgttgacgacaacggtgcgat
gggtcccggccccggtcaagacgatgccaatacgttgcgacactacgccttggcacttttagaattgccttatcg
tcctgataagaaatgtccgaccagctaaagacatcgcgtccaatcaaagcctagaaaatataggcgaagggac
gctaataagtctttcataagaccgcgcaaatctaaaaatatccttagattcacgatgcggcacttcggatgacttc
cgagcgagcctggaacctcagaaaaacgtctgagagataccgcgaggagatataccatgatgaacaaacatg
taaataaagtagctttaatcggagcgggttttgttggaagcagttatgcatttgcgttaattaaccaaggaatcaca
gatgagcttgtggtcattgatcgcaataaagaaaaagcaatgggcgatgtgatggatttaaaccacggaaaggc
gtttgcgccacaaccggtcaaaacatcttacggaacatatgaagactgcaaggatgctgatattgtctgcatttgc
gccggagcaaaccaaaaacctggtgagacacgccttgaattagtagaaaagaacttgaagattttcaaaggcat
cgttagtgaagtcatggcgagcggatttgacggcattttcttagtcgcgacaaatccggttgatatcctgacttac
gcaacatggaaattcagcggcctgccaaaagagcgggtgattggaagcggcacaacacttgattctgcgagat
tccgtttcatgctgagcgaatactttggcgcagcgcctcaaaacgtacacgcgcatattatcggagagcacggc
gacacagagcttcctgtttggagccacgcgaatgtcggcggtgtgccggtcagtgaactcgttgagaaaaacg
atgcgtacaaacaagaggagctggaccaaattgtagatgatgtgaaaaacgcagcttaccatatcattgagaaa
aaaggcgcgacttattatggggttgcgatgagtcttgctcgcattacaaaagccattcttcataatgaaaacagca
tattaactgtcagcacatatttggacgggcaatacggtgcagatgacgtgtacatcggtgtgccggctgtcgtga
atcgcggagggatcgcaggtatcactgagctgaacttaaatgagaaagaaaaagaacagttccttcacagcgc
cggcgtccttaaaaacattttaaaacctcattttgcagaacaaaaagttaactaatgcctggcggcagtagcgcg
gtggtcccacctgaccccatgccgaactcagaagtgaaacgccgtagcgccgatggtagtgtggggtctcccc
atgcgagagtagggaactgccaggcatcaaataaaacgaaaggctcagtcgaaagactgggcctttcgttttat
ctgttgtttgtcggtgaaactagtctgcaggaattcgatatcaagttaagccagccccgacacccgccaacaccc
gctgacgcgccctgacgggcttgtctgctcccggcatccgcttacagacaagctgtgaccgtctccgggagct
gcatgtgtcagaggttagaaaaactcatcgagcatcaaatgaaactgcaatttattcatatcaggattatcaatac
catatttttgaaaaagccgtttctgtaatgaaggagaaaactcaccgaggcagttccataggatggcaagatcct
ggtatcggtctgcgattccgactcgtccaacatcaatacaacctattaatttcccctcgtcaaaaataaggttatca
agtgagaaatcaccatgagtgacgactgaatccggtgagaatggcaaaagcttatgcatttctttccagacttgtt
caacaggccagccattacgctcgtcatcaaaatcactcgcatcaaccaaaccgttattcattcgtgattgcgcct
gagcgagacgaaatacgcgatcgctgttaaaaggacaattacaaacaggaatcgaatgcaaccggcgcagga
acactgccagcgcatcaacaatattttcacctgaatcaggatattcttctaatacctggaatgctgttttcccgggg
atcgcagtggtgagtaaccatgcatcatcaggagtacggataaaatgcttgatggtcggaagaggcataaattc
cgtcagccagtttagtctgaccatctcatctgtaacatcattggcaacgctacctttgccatgtttcagaaacaact
ctggcgcatcgggcttcccatacaatcgatagattgtcgcacctgattgcccgacattatcgcgagcccatttata
cccatataaatcagcatccatgttggaatttaatcgcggcctcgagcaagacgtttcccgttgaatatggctcata
acaccccttgtattactgtttatgtaagcagacagttttattgttcatgatgatatatttttatcttgtgcaatgtaacat
cagagattttgagacacaacgtggctttcccccccccccctgcaggtccgacacggggatggatggcgttccc
gatcatggtcctgcttgcttcgggagcgatactgagcgaagcaagtgcgtcgagcagtgcccgcttgttcctga
aatgccagtaaagcgctggctgctgaacccccagccggaactgaccccacaaggccctagcgtttgcaatgc
accaggtcatcattgacccaggcgtgttccaccaggccgctgcctcgcaactcttcgcaggcttcgccgacctg
ctcgcgccacttcttcacgcgggtggaatccgatccgcacatgaggcggaaggtttccagcttgagcgggtac
ggctcccggtgcgagctgaaatagtcgaacatccgtcgggccgtcggcgacagcttgcggtacttctcccatat
gaatttcgtgtagtggtcgccagcaaacagcacgacgatttcctcgtcgatcaggacctggcaacgggacgttt
tcttgccacggtccaggacgcggaagcggtgcagcagcgacaccgattccaggtgcccaacgcggtcggac
gtgaagcccatcgccgtcgcctgtaggcgcgacaggcattcctcggccttcgtgtaataccggccattgatcga
ccagcccaggtcctggcaaagctcgtagaacgtgaaggtgatcggctcgccgataggggtgcgcttcgcgta
ctccaacacctgctgccacaccagttcgtcatcgtcggcccgcagctcgacgccggtgtaggtgatcttcacgt
ccttgttgacgtggaaaatgaccttgttttgcagcgcctcgcgcgggattttcttgttgcgcgtggtgaacagggc
agagcgggccgtgtcgtttggcatcgctcgcatcgtgtccggccacggcgcaatatcgaacaaggaaagctg
catttccttgatctgctgcttcgtgtgtttcagcaacgcggcctgcttggcctcgctgacctgttttgccaggtcctc
gccggcggtttttcgcttcttggtcgtcatagttcctcgcgtgtcgatggtcatcgacttcgccaaacctgccgcct
cctgttcgagacgacgcgaacgctccacggcggccgatggcgcgggcagggcagggggagccagttgcac
gctgtcgcgctcgatcttggccgtagcttgctggaccatcgagccgacggactggaaggtttcgcggggcgca
cgcatgacggtgcggcttgcgatggtttcggcatcctcggcggaaaaccccgcgtcgatcagttcttgcctgtat
gccttccggtcaaacgtccgattcattcaccctccttgcgggattgccccgactcacgccggggcaatgtgccc
ttattcctgatttgacccgcctggtgccttggtgtccagataatccaccttatcggcaatgaagtcggtcccgtag
accgtctggccgtccttctcgtacttggtattccgaatcttgccctgcacgaataccagctccgcgaagtcgctct
tcttgatggagcgcatggggacgtgcttggcaatcacgcgcaccccccggccgttttagcggctaaaaaagtc
atggctctgccctcgggcggaccacgcccatcatgaccttgccaagctcgtcctgcttctcttcgatcttcgcca
gcagggcgaggatcgtggcatcaccgaaccgcgccgtgcgcgggtcgtcggtgagccagagtttcagcagg
ccgcccaggcggcccaggtcgccattgatgcgggccagctcgcggacgtgctcatagtccacgacgcccgt
gattttgtagccctggccgacggccagcaggtaggcctacaggctcatgccggccgccgccgccttttcctca
atcgctcttcgttcgtctggaaggcagtacaccttgataggtgggctgcccttcctggttggcttggtttcatcagc
catccgcttgccctcatctgttacgccggcggtagccggccagcctcgcagagcaggattcccgttgagcacc
gccaggtgcgaataagggacagtgaagaaggaacacccgctcgcggggggcctacttcacctatcctgccc
ggctgacgccgttggatacaccaaggaaagtctacacgaaccctttggcaaaatcctgtatatcgtgcgaaaaa
ggatggatataccgaaaaaatcgctataatgaccccgaagcagggttatgcagcggaaaagatccgtc

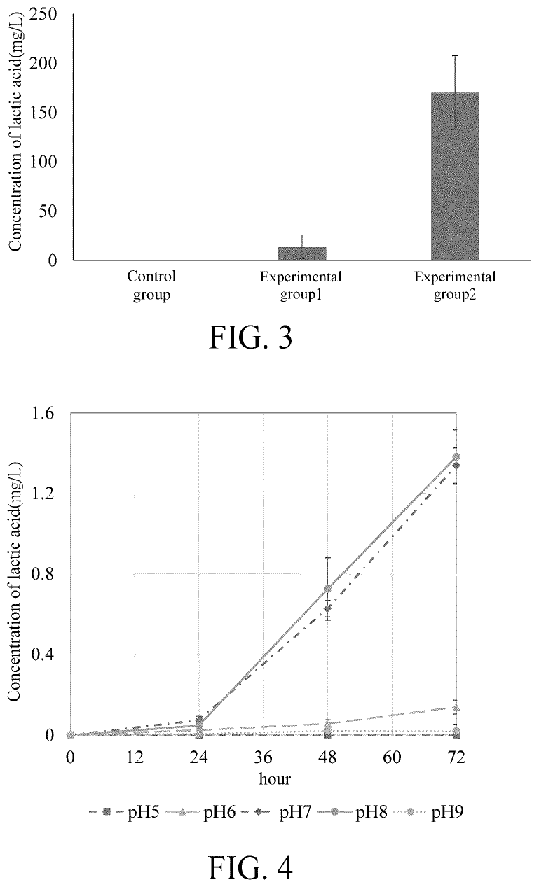

The experimental results are as shown in FIG. 3. The control strain that has not been added with the mutant lactate dehydrogenase gene ldh V39R at all still does not produce lactic acid after 24 hours of cultivation. The strain of Experimental group 1 (wild-type Methylobacterium extorquens containing ldh V39R gene) produces about 13.52 mg/L lactic acid, and the strain of Experimental group 2 (the NITE BP-04142 strain containing the ldh V39R gene in the example) produces about 170 mg/L of lactic acid. According to the above experimental data, on the premise that the mutant lactate dehydrogenase gene ldh V39R is added, the lactic acid yield of the NITE BP-04142 strain in the example can reach 13 times that of the wild-type Methylobacterium extorquens. Moreover, in other repeated test results conducted with the same process, the lactic acid yield of the NITE BP-04142 strain in this example can reach at least 10 times or more that of the wild-type Methylobacterium extorquens. Judging from the overall trend of the above test results, the NITE BP-04142 strain of this example is far superior to the wild type Methylobacterium extorquens with the addition of mutant lactate dehydrogenase gene ldh V39R in increasing lactate production through the mutant lactate dehydrogenase gene ldh V39R.

Test of Lactic Acid Yield for Methylobacterium extorquens Under Environments with Different pH Values

This test will further determine the lactic acid yield of the NITE BP-04142 strain in the example under various environments with different pH values. The test process proceeds as what follows.

First, the NITE BP-04142 strain of the example was prepared according to the aforementioned test process. The NITE BP-04142 strain was inoculated into 2 mL of the culture medium C and pre-cultured at 30° C. and 250 rpm for 2 to 3 days.

In addition, 20 mL of the culture medium B containing 50 μg/mL kanamycin were divided into five equal parts (4 ml), of which the pH values were adjusted with 1N hydrochloric acid aqueous solution and 1N sodium hydroxide aqueous solution to pH 5, pH 6, pH 7, pH 8 and pH 9 respectively. The culture medium B with pH 5 was used as Experimental group 1; the culture medium B with pH 6 was used as Experimental group 2; the culture medium B with pH 7 was used as Experimental group 3; the culture medium B with pH 8 was used as Experimental group 4; and the culture medium B with pH 6 was used as Experimental group 5.

Furthermore, after the pre-culture of the NITE BP-04142 strain was completed, the bacterial liquid was inoculated into the culture medium B of Experimental groups 1 to 5, and the initial OD₆₀₀ values of Experimental groups 1 to 5 were adjusted to 0.1. The fermentation broths of Experimental groups 1 to 5 were supplemented with 1% v/v methanol every 24 hours after the start of culture, and the pH values of the fermentation broths of Experimental groups 1 to 5 were adjusted to the preset pH values respectively at the time of methanol supplementation. The fermentation broths of Experimental groups 1 to 5 were sampled at every 12 hours and continued to ferment until 72 hours. The fermentation broth samples of Experimental groups 1 to 5 were analyzed for lactic acid content at each time point using the aforementioned liquid chromatography Shimadzu Prominence-I (LC2030C 3D) in conjunction with the Agilent HiPlex-H (700×7.7 mm) organic acid analysis column.

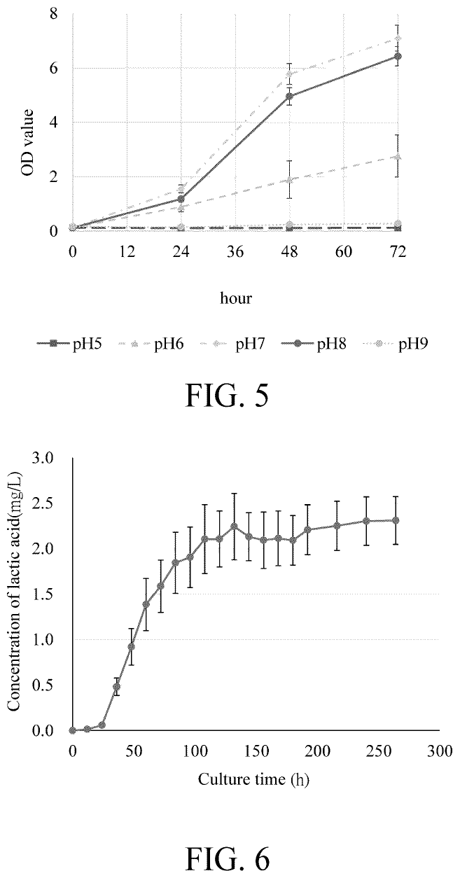

The test results are shown in FIG. 4. The lactic acid yields of Experimental groups 2 to 5 at the 72nd hour were 0.14 g/L, 1.3 g/L, 1.4 g/L and 0.02 g/L respectively. In Experimental group 1, the lactic acid yield could not be detected. According to the above results, it can be seen that the NITE BP-04142 strain has good lactic acid yield in an environment of pH 7 to pH 8. On the other hand, it can be observed from FIG. 5 that the growth of the NITE BP-04142 strain in Experimental group 2 (pH 6) and Experimental group 5 (pH 9) was inhibited, while in Experimental group 1 the NITE BP-04142 strain stopped growing. The growth conditions of Experimental group 3 (pH 7) and Experimental group 4 (pH 8) are good and there is no obvious difference. According to the above test results, the range of pH value of the culture medium in which the NITE BP-04142 strain can grow well is approximately between pH 6 and pH 9, preferably between pH 6.5 and pH 8.5, and more preferably between pH 7 and pH 8.

Test of Long-Term Lactic Acid Yield of Methylobacterium extorquens Under pH 7 Environment

According to the aforementioned test results, after testing the lactic acid yield of the NITE BP-04142 strain of the example under different pH environments, it is found that the NITE BP-04142 strain of the example has the best lactic acid yield in an environment from pH 7 to pH 8. This test will further test the changes in lactic acid yield of the NITE BP-04142 strain of the example during cultivation in a pH 7 environment for 300 hours. The testing process proceeds as what follows.

First, the NITE BP-04142 strain of the example was prepared according to the aforementioned test process. The NITE BP-04142 strain was inoculated into 2 mL of the culture medium C and pre-cultured at 30° C. and 250 rpm for 2 to 3 days. After the pre-culture was completed, the bacterial liquid was inoculated into 20 mL the culture medium B containing 50 μg/mL kanamycin, the initial OD 600 value of the fermentation broth was adjusted to 0.1, and the pH value of the fermentation broth was adjusted to pH 7 with 1 N NaOH solution. The fermentation broth was supplemented with methanol at a concentration of 1% v/v every 12 hours after the start of culture, and the pH value of the fermentation broth was adjusted to pH 7. The fermentation broth was sampled every 12 hours and continued to ferment for 264 hours. The samples were analyzed for lactic acid content at each time point using the liquid chromatography Shimadzu Prominence-I (LC2030C 3D) in conjunction with the Agilent HiPlex-H (700×7.7 mm) organic acid analysis column.

The test results are shown in FIG. 6. When the NITE BP-04142 strain is fermented to about the 108th hour, the lactic acid yield reaches 2.1 g/L, thereafter the yield of the NITE BP-04142 strain is continuously maintained at 2.1 g/L or above, and the yield of the NITE BP-04142 strain can reach 2.31 g/L at the 264th hour of fermentation. This test shows that when the strain of the example is in its optimum pH 7 growth environment, it can maintain high lactic acid production yield for a long time.

In this test, the culture medium B is used as the growth medium of NITE BP-04142 strain. In the formulation of the culture medium B, there is 7.9 g/L (i.e. 1% v/v) methanol. However, the test results show that the NITE BP-04142 strain has a better lactic acid yield under methanol conditions of 0.5% v/v to 1.5% v/v. In addition, it is observed in other test results that when the methanol concentration in the culture environment is increased to above 2% v/v, the NITE BP-04142 strain will begin to show growth inhibition. In other words, the NITE BP-04142 strain has better lactic acid yield in the following concentration range: 0.5% v/v, 0.6% v/v, 0.7% v/v, 0.8% v/v, 0.9% v/v, 1.0% v/v, 1.1% v/v, 1.2% v/v, 1.3% v/v, 1.4% v/v and 1.5% v/v. Meanwhile, when the methanol concentration in the culture environment is lower than 2% v/v, the NITE BP-04142 strain can grow and ferment to produce lactic acid. In addition, it is observed in another test result that when the methanol concentration in the culture environment is below 2% v/v, the methanol concentration can be adjusted between 1 g/L and 40 g/L.

The culture medium B used for the growth of the NITE BP-04142 strain in this test uses 1 g/L (NH₄)₂SO₄ as the nitrogen source. The carbon content of the culture medium B is 2.9625 g/L (7.9 g/L methanol), and the nitrogen content of the culture medium B is 0.2119 g/L (1 g/L (NH₄)₂SO₄). Therefore, the carbon/nitrogen ratio of the culture medium B is 2.9625/0.2119, which is calculated to be approximately 14. It is observed in other test results that when the carbon/nitrogen ratio of the culture medium is between 3.5 and 21, it is suitable for the NITE BP-04142 strain to grow and produce lactic acid. That is, the NITE BP-04142 strain is suitable to grow within the following range of carbon/nitrogen ratio: 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21. In addition, the carbon/nitrogen ratios of the NITE BP-04142 strain between 13 and 15 give good yields. Moreover, it is observed in another test result that the nitrogen source concentration in the culture medium B can be adjusted between 0.1 g/L and 40 g/L, for example, the yeast extract or peptone having a concentration between 0.1 g/L and 0.5 g/L.

In this test, the NITE BP-04142 strain is grown and cultured in an environment of 30° C. It is observed in other test results that the NITE BP-04142 strain can grow and produce lactic acid in an environment of 20° C. to 30° C., such as 20° C., 21° C., 22° C., 23° C., 24° C., 25° C., 26° C., 27° C., 28° C., 29° C. or 30° C.; and the optimal growth and fermentation environment for NITE BP-04142 strain is 28° C. to 30° C.

Test of Long-Term Lactic Acid Yield of Methylobacterium extorquens Under Various Environment

This test will further test the changes in lactic acid yield of the NITE BP-04142 strain of the example during cultivation under various environment, such as pH value, methanol concentration, concentration of (NH₄)₂SO₄ as nitrogen content, concentration of (NH₄)₂HPO₄ as alternative nitrogen content, and the presence of yeast extract and peptone. The testing process proceeds as what follows.

First, the NITE BP-04142 strain of the example was prepared according to the aforementioned test process. Thirty two micro fermentation tanks (BioLector Pro (M2P labs)) were also prepared, each fermentation tank was loaded in a culture medium which was based on the culture medium B and changed one condition or one content of the original culture medium B. The above fermentation tanks were numbered as groups 1 to 32.

The groups 1-7 changed the pH value of the culture medium B and were used for testing the effect of changing pH value on the NITE BP-04142 strain. The pH values of groups 1-7 were shown in Table 4.

TABLE 4
The pH value of groups 1-7

| Group 1 | Group 2 | Group 3 | Group 4 | Group 5 | Group 6 | Group 7 |
|---|---|---|---|---|---|---|
| pH 4 | pH 5 | pH 6 | pH 7 | pH 8 | pH 9 | pH 10 |

The groups 8-14 changed the methanol concentration of the culture medium B and were used for testing the effect of changing the methanol concentration on the NITE BP-04142 strain. The methanol concentration of groups 8-14 were shown in Table 5.

TABLE 5
The methanol concentration of groups 8-14

| Group 8 | Group 9 | Group 10 | Group 11 | Group 12 | Group 13 | Group 14 |
|---|---|---|---|---|---|---|
| 1 g/L | 2 g/L | 5 g/L | 10 g/L | 20 g/L | 30 g/L | 40 g/L |
| MeOH | MeOH | MeOH | MeOH | MeOH | MeOH | MeOH |

The groups 15-21 changed the concentration of (NH₄)₂SO₄ and were used for testing the effect of changing the concentration of (NH₄)₂SO₄ on the NITE BP-04142 strain. The concentration of (NH₄)₂SO₄ of groups 15-21 were shown in Table 6.

TABLE 6
The concentration of (NH₄)₂SO₄ of groups 15-21

| Group 15 | Group 16 | Group 17 | Group 18 | Group 19 | Group 20 | Group 21 |
|---|---|---|---|---|---|---|
| 0.1 g/L | 0.5 g/L | 1 g/L | 5 g/L | 10 g/L | 20 g/L | 40 g/L |
| (NH₄)₂SO₄ | (NH₄)₂SO₄ | (NH₄)₂SO₄ | (NH₄)₂SO₄ | (NH₄)₂SO₄ | (NH₄)₂SO₄ | (NH₄)₂SO₄ |

The groups 22-28 changed the nitrogen content of the culture medium B into (NH₄)₂HPO₄ with different concentrations and were used for testing the effect of the alternative nitrogen content with different concentrations on the NITE BP-04142 strain. The concentration of (NH₄)₂HPO₄ of groups 22-28 were shown in Table 7.

TABLE 7
The concentration of (NH₄)₂HPO₄ of groups 22-28

| Group 22 | Group 23 | Group 24 | Group 25 | Group 26 | Group 27 | Group 28 |
|---|---|---|---|---|---|---|
| 0.1 g/L | 0.5 g/L | 1 g/L | 5 g/L | 10 g/L | 20 g/L | 40 g/L |
| (NH₄)₂PO₄ | (NH₄)₂PO₄ | (NH₄)₂PO₄ | (NH₄)₂PO₄ | (NH₄)₂PO₄ | (NH₄)₂PO₄ | (NH₄)₂PO₄ |

The groups 29-32 additionally add yeast extract and peptone with different concentrations into the culture medium and were used for testing the effect of the presence of yeast extract and peptone on the NITE BP-04142 strain, the concentrations of methanol of the groups 29-32 were adjusted corresponding to the concentrations of yeast extract and peptone in each group. The concentration of yeast extract and peptone of groups 29-32 were shown in Table 8.

TABLE 8
The concentration of yeast extract (YE)
and peptone (PT) of groups 29-32

| Group 29 | Group 30 | Group 31 | Group 32 |
|---|---|---|---|
| 5 g/L MeOH/ | 10 g/L MeOH/ | 15 g/L MeOH/ | 20 g/L MeOH/ |
| 0.2 g/L YE | 0.5 g/L YE | 1.0 g/L YE | 1.5 g/L YE |
| and 0.2 g/L | and 0.5 g/L | and 1.0 g/L PT | and 1.5 g/L PT |
| PT | PT | | |

After the fermentation tanks of groups 1-32 loaded in their corresponding culture medium were prepared, 2 mL of the NITE BP-04142 strain was inoculated into each of the groups 1-32 to prepare fermentation broths of the groups 1-32. The fermentation broths of the groups 1-32 fermented for 96 hours at a rotation speed of 1200 rpm and under 30° C. The fermentation broths of the groups 1-32 were supplemented with methanol at a concentration of 4 g/L per day after the start of fermentation, and the pH value of the fermentation broths of the groups 1-32 were adjusted by 4N NaOH to maintain their pH value at the initial pH value.

The fermentation broths of the groups 1-32 were sampled at 72th hour and 96th hour. The samples broths of the groups 1-32 were analyzed for OD₆₀₀ value and lactic acid yield.

The OD₆₀₀ value and lactic acid yield of the groups 1-32 were shown in the following tables 9-13. It is demonstrated that the NITE BP-04142 strain can grow and ferment to produce lactic acid at least in an environment from pH 4 to pH 9. Meanwhile, the presence of yeast extract and peptone in the culture medium is also proven that it can enhance the lactic acid yield of the NITE BP-04142 strain.

TABLE 9
The OD₆₀₀ value and lactic acid yield of the groups 1-7 at different times

| Group | 1 | 2 | 3 | 4 | 5 | 6 | 7 |
|---|---|---|---|---|---|---|---|
| 72 hr OD₆₀₀ | 0.7 | 0.92 | 1.08 | 17.22 | 13.8 | 0.02 | 0.02 |
| value | | | | | | | |
| 72 hr lactic | 0.118017 | 0.135368 | 0.266796 | 1.157117 | 1.120447 | 0.023066 | 0 |
| acid yield | | | | | | | |
| (g/L) | | | | | | | |
| 96 hr OD₆₀₀ | 0.52 | 0.66 | 0.9 | 16.14 | 16.98 | 0.24 | 0.26 |
| value | | | | | | | |
| 96 hr lactic | 0.116993 | 0.215829 | 0.283145 | 1.343165 | 2.165484 | 0.013892 | 0.05111 |
| acid yield | | | | | | | |
| (g/L) | | | | | | | |

TABLE 10
The OD₆₀₀ value and lactic acid yield of the groups 8-14 at different times

| Group | 8 | 9 | 10 | 11 | 12 | 13 | 14 |
|---|---|---|---|---|---|---|---|
| 72 hr OD₆₀₀ | 19.02 | 18.98 | 18.72 | 18.56 | 15.26 | 0.02 | 0.02 |
| value | | | | | | | |
| 72 hr lactic | 1.670164 | 1.685166 | 1.902606 | 1.73359 | 1.501304 | 0 | 0 |
| acid yield | | | | | | | |
| (g/L) | | | | | | | |
| 96 hr OD₆₀₀ | 13.86 | 20.7 | 19.92 | 19.16 | 14.8 | 0.28 | 0.14 |
| value | | | | | | | |
| 96 hr lactic | 1.433722 | 2.208778 | 2.310989 | 2.218203 | 1.906365 | 0.075312 | 0 |
| acid yield | | | | | | | |
| (g/L) | | | | | | | |

TABLE 11
The OD₆₀₀ value and lactic acid yield of the groups 15-21 at different times

| Group | 15 | 16 | 17 | 18 | 19 | 20 | 21 |
|---|---|---|---|---|---|---|---|
| 72 hr OD₆₀₀ | 1.82 | 8.68 | 17.22 | 12.18 | 0.22 | 0.04 | 0.02 |
| value | | | | | | | |
| 72 hr lactic | 0.364113 | 1.243215 | 1.667842 | 0.857478 | 0 | 0 | 0 |
| acid yield | | | | | | | |
| (g/L) | | | | | | | |
| 96 hr OD₆₀₀ | 1.56 | 7.96 | 18.5 | 13.42 | 0.2 | 0.26 | 0.24 |
| value | | | | | | | |
| 96 hr lactic | 0.494561 | 1.479259 | 2.232285 | 1.388297 | 0.07513 | 0 | 0 |
| acid yield | | | | | | | |
| (g/L) | | | | | | | |

TABLE 12
The OD₆₀₀ value and lactic acid yield of the groups 22-28 at different times

| Group | 22 | 23 | 24 | 25 | 26 | 27 | 28 |
|---|---|---|---|---|---|---|---|
| 72 hr OD₆₀₀ | 2.16 | 8.1 | 15.36 | 10 | 0.18 | 0.06 | 0.04 |
| value | | | | | | | |
| 72 hr lactic | 0.37132 | 1.173716 | 1.453975 | 0.600416 | 0.033922 | 0 | 0 |
| acid yield | | | | | | | |
| (g/L) | | | | | | | |
| 96 hr OD₆₀₀ | 1.68 | 7.66 | 15.94 | 12.56 | 0.1 | 0.22 | 0.12 |
| value | | | | | | | |
| 96 hr lactic | 0.44147 | 1.362167 | 1.848672 | 0.895096 | 0.096685 | 0 | 0 |
| acid yield | | | | | | | |
| (g/L) | | | | | | | |

TABLE 13
The OD₆₀₀ value and lactic acid yield
of the groups 29-32 at different times

| Group | 29 | 30 | 31 | 32 |
|---|---|---|---|---|
| 72 hr OD₆₀₀ value | 19.98 | 15.48 | 24.2 | 19.54 |
| 72 hr lactic acid yield | 2.27 | 2.25 | 1.42 | 3.23 |
| (g/L) | | | | |
| 96 hr OD₆₀₀ value | 19 | 14.26 | 28.2 | 20.84 |
| 96 hr lactic acid yield | 2.66 | 2.97 | 2.31 | 4.04 |
| (g/L) | | | | |

As mentioned above, the NITE BP-04142 strain of the example improves its lactic acid yield by knocking out the celABC gene and the lldEFG gene, thereby providing a strain that can use methanol to produce lactic acid, and providing a lactic acid production method that can uses non-food crop raw materials to produce lactic acid, so as to solve the problem that conventional lactic acid production method will crowd out the demand for food consumption. At the same time, when the NITE BP-04142 strain expresses the mutant lactate dehydrogenase gene ldh V39R in vivo, it can significantly increase lactic acid yield, compared with the wild-type Methylobacterium extorquens that also expresses the mutant lactate dehydrogenase gene ldh V39R. In addition, when the NITE BP-04142 strain is in the growth environment of pH 7 to pH 8, it has better lactic acid yield and can maintain high lactic acid yield for a long time.

The present invention has been disclosed above with preferred embodiments. However, those skilled in the art should understand that the embodiments are only used to illustrate the present invention and should not be interpreted as limiting the scope of the present invention. It should be noted that any changes and substitutions that are equivalent to the embodiments should be considered to be within the scope of the present invention. Therefore, the protection scope of the present invention shall be determined by the appended claims.

What is claimed is:

1. A Methylobacterium extorquens, capable of fermenting methanol to produce lactic acid, wherein the Methylobacterium extorquens is deposited at Japan National Institute of Technology and Evaluation with a accession number NITE BP-04142.

2. A method for producing lactic acid, comprising:
(a) providing a Methylobacterium extorquens capable of fermenting methanol to produce lactic acid, wherein the Methylobacterium extorquens is deposited at Japan National Institute of Technology and Evaluation with a accession number NITE BP-04142;
(b) inoculating the Methylobacterium extorquens in a methanol culture medium containing methanol at a concentration of below 2% v/v, wherein the methanol culture medium has a C/N ratio between 3.5 and 21 and a pH value between pH 4 and pH 9; and
(c) allowing the Methylobacterium extorquens to ferment the methanol in the methanol culture medium to produce lactic acid under 20° C. to 30° C.

3. The method of claim 2, wherein in step (a), the Methylobacterium extorquens is caused to express lactate dehydrogenase gene ldh V39R of Bacillus subtilis in vivo.

4. The method of claim 3, wherein the concentration of the methanol in the methanol culture medium ranges from 0.5% to 1%.

5. The method of claim 3, wherein the pH value of the methanol culture medium ranges from pH 7 to pH 8.

6. The method of claim 3, wherein the C/N ratio of the methanol culture medium ranges from 13 to 15.

7. The method of claim 2, wherein the pH value of the methanol culture medium ranges from pH 7 to pH 8.

8. The method of claim 7, wherein the concentration of the methanol in the methanol culture medium ranges from 0.5% to 1%.

9. The method of claim 2, wherein the concentration of the methanol in the methanol culture medium ranges from 0.5% to 1%.

10. The method of claim 2, wherein the C/N ratio of the methanol culture medium ranges from 13 to 15.

11. The method of claim 2, wherein the methanol culture medium further contains yeast extract and peptone.


SEQUENCE LISTING (22 sequences)

SEQ ID NO: 1 - DNA, 5433 bp, synthetic construct
  source 1..5433: mol_type = other DNA
ggatccagcttatcgataccgcgggccctacgtacgcgtgttaaccggtgagctcactagaggatccagccgaccaggctttccacgcccgcgtgccgctccatgtcgttcgcgcggttctcggaaacgcgctgccgcgtttcgtgattgtcacgctcaagcccgtagtcccgttcgagcgtcgcgcagaggtcagcgagggcgcggtaggcccgatacggctcatggatggtgtttcgggtcgggtgaatcttgttgatggcgatatggatgtgcaggttgtcggtgtcgtgatgcacggcactgacgcgctgatgctcggcgaagccaagcccagcgcagatgcggtcctcaatcgcgcgcaacgtctccgcgtcgggcttctctcccgcgcggaagctaaccagcaggtgataggtcttgtcggcctcggaacgggtgttgccgtgctgggtcgccatcacctcggccatgacagcgggcagggtgtttgcctcgcagttcgtgacgcgcacgtgacccaggcgctcggtcttgccttgctcgtcggtgatgtacttcaccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgaggcggcgttagcgtccggctatgcgctcgtcaccatttgggagcacatgcgggaaacggggaaggtcaagttctcctacgagacgttccgctcgcacgccaggcggcacatcaaggccaagcccgccgatgtgcccgcaccgcaggccaaggctgcggaacccgcgccggcacccaagacgccggagccacggcggccgaagcaggggggcaaggctgaaaagccggcccccgctgcggccccgaccggcttcaccttcaacccaacaccggacaaaaaggatccccaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctagatctgaattcagctgtacaattggtaccatggatgcatatggcggccgc

SEQ ID NO: 2 - DNA, 1000 bp, synthetic construct
  source 1..1000: mol_type = other DNA
tacaccggcgcgcaggcgcaggcggtggacgacagctgggcggtggccgtcgatggctcgaccagcatcacctccaattcctcggccttcggctccgtcaccgtgacgacggcactcggcagcccgccgacggtgagcggcgcgcgcgtgcgcgtcgaggccgtggccggcacctactcctatcccggacaaggcgtcggcgcccgcgtcaccggctaccagcaggagggctcgctgctcaccggctacgagtggatctggcgggaggcggcactcgccggctatgtcggcttcaacgtccgcagcaaccagctctcgatccccgaccccggcaacccggtcgtcggaacgggagtgggcctgaaggtcgccggcaacttctacgcaacgccgaccgaccggaccatggtctcggcctacggctcctactcgaccaagttcaacgcctactattcccgtttccgcgtcggctacatggtcgccgacggcgtctatatcggcccggaggcgctgttcctcggcgacgacttcttccgccagtaccgcgtcggcgcccatctctcgggtttgagcttcggccccgtccagatgtcgctcgcggcgggctacgtgcgggaccgcgtccagggcacgggctactattccagcatcgaggcgcgcgcgaacttttagagcgcattccgacggcgagcaggccgccgctcgcggcggcggccgaagtcaagcgcttggatcaatgcggacaatccgccgccttgtctccgcgtctcacggcaaagtactgctgttccaagcgaagaatggccctcatatcaggaagacatccctcgccatcgcatgtcatttccgcgaaattcctgccgaatcttcggacattttctacggatgacgctacgctctgcatcgcaaaatcaaccttgttcgcagagcgggacggcggcgtcgtgaacgcatccggaaatcgtcgtattttaatgatggatccgtccacgaggcaggg

SEQ ID NO: 3 - DNA, 1000 bp, synthetic construct
  source 1..1000: mol_type = other DNA
cggcggaagaaagcgcgttgacactaagacctagagacgccggcctgatgcaatcaggtcggatacggctctaggacgatcgatgggacgagcactctgagacccgccatggaattcggaccttacgcccccttcatcgtcggttcctacggcttcgcggccctggtgctcggcgccctggccctcaacggctggtgggatgggcggacgcagcgccgcgcgctcgccgagctgcaggacgagcggggaggtcgctcgtgagcgcgcagatcccgcacgcgaccccgcccggtaccggggagcgcccccgccgctcgctcctcgcgctgctaccggtgctcgtcttcgccatcttggccgtcgccttcctggtccggctgcgctcgggcgccgatcctgcggctttgccctcggcgttgatcggcaaaccggtccccgctttcgcgctggagccggtgccgggcctccaggccgccggccggccggtgcccggtttgtcgagtgccgacctcaaggggcaggtgaccgtcctcaacatcttcgcctcgtggtgcgccccctgccagatcgagcatccgatgctgatgcggctcgcgcaggagcccggcatccgcctcgtcggcatcgactacaaggatcccggcgacgccggacggcgctggctggagcgcaacggcctgccgttcgcggcggtcggcgccgacacgagcggccgggccggaatcgacctcggcgtctacggcgtgccggagaccttcatcatcggtcccgaaggccgcatccgcgacaagctcgtcggaatcctgacgccggaaaactacgcgagcgtgctggagcgtattcgcgccgccggacggaccgcccccgtgaccaactgaccgggatcaggcggcacgcttggaaaccggggatcggaggcgtatgcagcccctgtccgcttgaaaccgttccaaggtggagaagcgggccaaccgtctcaaatcacgatcagaagagcgcc

SEQ ID NO: 4 - DNA, 584 bp, synthetic construct
  source 1..584: mol_type = other DNA
ataacttcgtatagcatacattatacgaagttatctagacctgcaggggggggggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgt

SEQ ID NO: 5 - DNA, 771 bp, synthetic construct
  source 1..771: mol_type = other DNA
gttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgcccccccccccctgcaggtcctagataacttcgtatagcatacattatacgaagttat

SEQ ID NO: 6 - DNA, 1000 bp, synthetic construct
  source 1..1000: mol_type = other DNA
gatcgcgtcttctcggcctcctcgcggtactcccggccctgctcgtcgtcggcccggccctggcgcagaccgcgccggtcctgcgcctgcccggcgccggtccggtcgcgggcgtcgtcgagacgcgggtgcgcgatgggttcgaccagcgcatccgcttcgagggcggcgacggccgcaaccatgccgaggtagccctgcgcaacgagaccggcgatctcctgctcgcgttcccgccgcggctggccaagccgagccagttcggcatcgacggcgagatcgcggtccgctttccggggcaggtcctgcgcgtggtcgcgccgcggcgcaacggctacgggccgatcggcctcgcgctcgggaccgactgcctctacgcgtggcaatggttcgagcgggcgagcgcggtccagcgcgccgcgagccgcggcggcctgttcgacacgtcggtctcgccaaccgcgggccggcgcgccctctccctccggattcggctgtgccgcacggcgcaggcgagtctcgacgatctcgtcgccgccatcgagcggatgaccatctctcttcccggcgagcccggagcacggtccgctcccgcgcgaccacgtccctccgccgtccggcgcgaaagacccgcgcccgtgaagcggcaggccgcgaggcccacccccagcgagccgcccgccgaggctgcaccgaagccagcaccggccgccccgccgcaggccgcgccgccgctccgtccgccgacgccaacggcgccggacagcggcggccgccgctacctcgccccaacgacgccgcagggcgccgccccggcctccgaaccgccggtcgcgagtccggcgccgggagggacgcagcgctacatcaccgatgtgccgaaaccggcagagggcgcgggcggcggcctgatgccgagcccggctccgggccgctcgaccggcgagagcctgtcgcgcgatctccccgccgaggcgtacaagcccgccccaagccgcga

SEQ ID NO: 7 - DNA, 1000 bp, synthetic construct
  source 1..1000: mol_type = other DNA
aagcggagccgaacggatctctgcgcgtttgcggcaaaacgcctcgctcgaacggggcgtcttcccgcaaaattcggagccgtccccgtgcgattcaagctcggttgcagtcttgcctatgaggtcaaggcgcccaccaccttcatcttcaatgtcgaggtcgcgaagctgcaaagcctcgacatcgcccgcgaaagcctgaccctggcgccggacctggcgcggcgcgtctacgtcacgccggacctcaagaaccgctatctcggcgtgaacgtgcagccgggcccgttctcgctcgaatacaatgccgaggtggatctggccgtcgtgcgggccgatcctgcgacgatctccgagacgccggtctccgaactccccctcgacatcctccccttcctgctgccgagccgcttcgtctcgtcggaccggctcgccgccttcgcccaagccgagttcggcgaccttccgaagggccaccagcgcgtcaacgcgatctgcaactggatccatgacaacatcgcctatcggcgtggttcgagcgacggcgagacgacggccgacgagagcctgctgatgcgtgccggcgtgtgccgcgacttcgcccatctcggcgccgccttctgccgcgggctcggcatcccggcccggttcgtgagctgctacgcctacggcctcgtgcccagcgacttccacgccgtgttcgaggcctatctcgatgggcgctggtggctgttcgacgcgacgcgccaagcccatctcgacggtctcgtgcgcatcggcatcgggcgggatgccgccgaaatcgccttctcgacgccctacggcgagatgcagccgaccggtatggagatccgcatcgatcgcgccgacggaggcccggagccgatggagcgcacggtcgatgcgatctcgacggaggagccggctccgcatgccggcgcggcctgaccgaggggcctcacgatcgacgccctacccggcgaatccggtggcccgagcg

SEQ ID NO: 8 - DNA, 25 bp, synthetic construct
  source 1..25: mol_type = other DNA
ggatccagcttatcgataccgcggg

SEQ ID NO: 9 - DNA, 25 bp, synthetic construct
  source 1..25: mol_type = other DNA
gcggccgccatatgcatccatggta

SEQ ID NO: 10 - DNA, 41 bp, synthetic construct
  source 1..41: mol_type = other DNA
ataacttcgtatagcatacattatacgaagttatctagacc

SEQ ID NO: 11 - DNA, 28 bp, synthetic construct
  source 1..28: mol_type = other DNA
acaattacaaacaggaatcgaatgcaac

SEQ ID NO: 12 - DNA, 28 bp, synthetic construct
  source 1..28: mol_type = other DNA
gttgcattcgattcctgtttgtaattgt

SEQ ID NO: 13 - DNA, 39 bp, synthetic construct
  source 1..39: mol_type = other DNA
ataacttcgtataatgtatgctatacgaagttatctagg

SEQ ID NO: 14 - DNA, 45 bp, synthetic construct
  source 1..45: mol_type = other DNA
tggatgcatatggcggccgctacaccggcgcgcaggcgcaggcgg

SEQ ID NO: 15 - DNA, 45 bp, synthetic construct
  source 1..45: mol_type = other DNA
tgtatgctatacgaagttatccctgcctcgtggacggatccatca

SEQ ID NO: 16 - DNA, 45 bp, synthetic construct
  source 1..45: mol_type = other DNA
catacattatacgaagttatgatcgcgtcttctcggcctcctcgc

SEQ ID NO: 17 - DNA, 45 bp, synthetic construct
  source 1..45: mol_type = other DNA
ggtatcgataagctggatcctcgcggcttggggcgggcttgtacg

SEQ ID NO: 18 - DNA, 39 bp, synthetic construct
  source 1..39: mol_type = other DNA
tggatgcatatggcggccgccggcggaagaaagcgcgtt

SEQ ID NO: 19 - DNA, 43 bp, synthetic construct
  source 1..43: mol_type = other DNA
tgtatgctatacgaagttatggcgctcttctgatcgtgatttg

SEQ ID NO: 20 - DNA, 40 bp, synthetic construct
  source 1..40: mol_type = other DNA
catacattatacgaagttataagcggagccgaacggatct

SEQ ID NO: 21 - DNA, 35 bp, synthetic construct
  source 1..35: mol_type = other DNA
ggtatcgataagctggatcccgctcgggccaccgg

SEQ ID NO: 22 - DNA, 6820 bp, synthetic construct
  source 1..6820: mol_type = other DNA
gaccctttccgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagccaagctgaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctcccgcttggtcgggccgcttcgcgagggcccgttgacgacaacggtgcgatgggtcccggccccggtcaagacgatgccaatacgttgcgacactacgccttggcacttttagaattgccttatcgtcctgataagaaatgtccgaccagctaaagacatcgcgtccaatcaaagcctagaaaatataggcgaagggacgctaataagtctttcataagaccgcgcaaatctaaaaatatccttagattcacgatgcggcacttcggatgacttccgagcgagcctggaacctcagaaaaacgtctgagagataccgcgaggagatataccatgatgaacaaacatgtaaataaagtagctttaatcggagcgggttttgttggaagcagttatgcatttgcgttaattaaccaaggaatcacagatgagcttgtggtcattgatcgcaataaagaaaaagcaatgggcgatgtgatggatttaaaccacggaaaggcgtttgcgccacaaccggtcaaaacatcttacggaacatatgaagactgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacaaatccggttgatatcctgacttacgcaacatggaaattcagcggcctgccaaaagagcgggtgattggaagcggcacaacacttgattctgcgagattccgtttcatgctgagcgaatactttggcgcagcgcctcaaaacgtacacgcgcatattatcggagagcacggcgacacagagcttcctgtttggagccacgcgaatgtcggcggtgtgccggtcagtgaactcgttgagaaaaacgatgcgtacaaacaagaggagctggaccaaattgtagatgatgtgaaaaacgcagcttaccatatcattgagaaaaaaggcgcgacttattatggggttgcgatgagtcttgctcgcattacaaaagccattcttcataatgaaaacagcatattaactgtcagcacatatttggacgggcaatacggtgcagatgacgtgtacatcggtgtgccggctgtcgtgaatcgcggagggatcgcaggtatcactgagctgaacttaaatgagaaagaaaaagaacagttccttcacagcgccggcgtccttaaaaacattttaaaacctcattttgcagaacaaaaagttaactaatgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaaactagtctgcaggaattcgatatcaagttaagccagccccgacacccgccaacacccgctgacgcgccctgacgggcttgtctgctcccggcatccgcttacagacaagctgtgaccgtctccgggagctgcatgtgtcagaggttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtccgacacggggatggatggcgttcccgatcatggtcctgcttgcttcgggagcgatactgagcgaagcaagtgcgtcgagcagtgcccgcttgttcctgaaatgccagtaaagcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagctccgcgaagtcgctcttcttgatggagcgcatggggacgtgcttggcaatcacgcgcaccccccggccgttttagcggctaaaaaagtcatggctctgccctcgggcggaccacgcccatcatgaccttgccaagctcgtcctgcttctcttcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggcctacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagatccgtc